(19) United States
(12) Patent Application Publication
(10) Pub. No.: US 2009/0299080 A1
(43) Pub. Date: Dec. 3, 2009

(54) Photochromic Compound

(75) Inventors: Yasushi Yokoyama, Yokohama (JP); Takashi Ubukata, Yokohama (JP); Tsuyoshi Gushiken, Yokohama (JP)

Correspondence Address: CROWELL & MORING LLP;INTELLECTUAL PROPERTY GROUP, P.O. BOX 14300, WASHINGTON, DC, 20044-4300, US

(73) Assignees: YOKOHAMA NATIONAL UNIVERSITY, NATIONAL UNIVERSITY CORPORATION, Yokohama (JP); CHEVRON U.S.A. INC., San Ramon, CA (US)

(21) Appl. No.: 12/282,232

(22) PCT Filed: Mar. 8, 2007
(86) PCT No.: PCT/JP2007/054515

§ 371 Date: Feb. 27, 2009

(30) Foreign Application Priority Data
Mar. 9, 2006 (JP) 2006-063829

(51) Int. Cl. C07D 333/78

(52) U.S. Cl. 549/41

(57) ABSTRACT

[Problem] To provide a photochromic compound that is rapidly colored upon irradiation with ultraviolet light, and is rapidly decolored rapidly upon heat application after termination of irradiation with ultraviolet light, and to provide a process for producing the same.

[Means for Solving Problems] A photochromic compound represented by general formula (I). R¹, R² and R³ are each a hydrogen atom; a functional group containing an atom in groups 15-16 of the periodic table; a halogen atom; a hydrocarbon group optionally substituted with said functional group or a halogen atom; or an optionally substituted heterocyclic group. R¹ and R² as well as R² and R³ may be bonded to each other to form an optionally substituted carbon or heterocyclic ring. Z is a polymantylidene group. W is a divalent organic group that forms an optionally substituted carbon aromatic or heterocyclic aromatic ring together with the carbon-carbon double bond to which it is bonded.

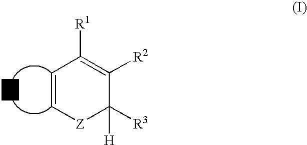

### TECHNICAL FIELD

The present invention relates to a novel photochromic compound. More specifically, it relates to a photochromic compound that has a bulky polymantylidene structure, and has a superior rate of response to ultraviolet light and a superior thermal decoloration rate.

### BACKGROUND ART

Photochromic compounds are compounds that can be reversibly switched between a colorless state and a colored state by irradiation with ultraviolet light, being used as photochromic materials, light-adjusting materials and the like, and applications as optical recording materials and the like are conceivable.

Among these, compounds wherein the coloration due to light is returned thermally to the colorless state are particularly suited as the light-adjusting materials for lenses for photochromic sunglasses, light-adjusting window glass and the like, as well as photochromic design materials.

Among these, Patent Reference 1 reports that fulgide compounds and fulgimide compounds having an adamantylidene group represented by general formula (R-I) are readily colored even by irradiation with sunlight.

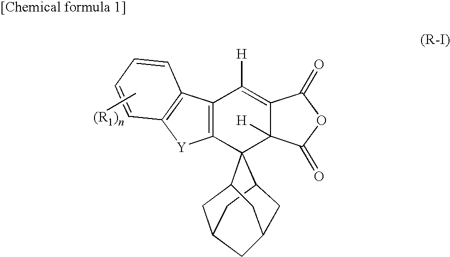

In addition, various improvements have been studied. For example, Patent Reference 2 discloses a photochromic compound wherein a norbornylidene group is used instead of the adamantylidene group.

In addition, Patent Reference 3 reports that by introducing various acid imide groups instead of the acid anhydride groups in general formula (R-I) above, the reversible durability in repeated coloration and discoloration is improved.

However, even with these improvements, a compound that is sufficiently satisfactory with respect to photoreactivity and thermoreactivity has not been obtained.

[Patent Reference 1] JP-A-S60-155179

[Patent Reference 2] JP-A-S64-40593

[Patent Reference 3] JP-A-H2-28154

### DISCLOSURE OF THE INVENTION

### Problems that the Invention is to Solve

Accordingly, an object of the present invention is to provide a photochromic compound that is rapidly colored by irradiation with ultraviolet light, and that is decolored rapidly upon the application of heat after the radiation of ultraviolet light is halted.

### Means of Solving the Problems

As a result of diligent research into the structure of fulgide compounds, the present inventors found that the aforementioned object can be achieved by introducing specific substituents into fulgide compounds, and as a result of proceeding with further research based on this knowledge, the present invention was achieved.

Thus, the present invention provides a photochromic compound represented by general formula (I).

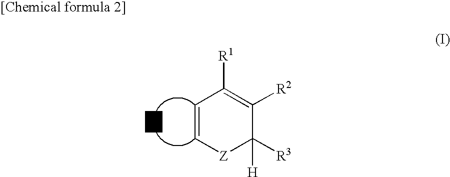

(In general formula (I), R¹, R² and R³ are each a hydrogen atom; a functional group containing an atom in groups 15-16 of the periodic table; a halogen atom; a hydrocarbon group optionally substituted with said functional group or a halogen atom; or an optionally substituted heterocyclic group.

R¹ and R² as well as R² and R³ may be bonded to each other to form an optionally substituted carbon or heterocyclic ring.

Z is a polymantylidene group.

W is a divalent organic group that forms an optionally substituted carbon aromatic or heterocyclic aromatic ring together with the carbon-carbon double bond to which it is bonded.)

The photochromic compound represented by general formula (I) is preferably represented by general formula (II).

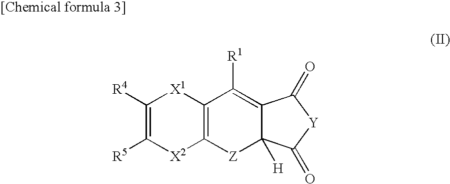

{In general formula (II), R¹ is a hydrogen atom; a functional group containing an atom in groups 15-16 of the periodic table; a halogen atom; a hydrocarbon group optionally substituted with said functional group or a halogen atom; or an optionally substituted heterocyclic group.

Y is an oxygen atom, a sulfur atom or >NR⁶. R⁶ is a hydrogen atom or an optionally substituted hydrocarbyl or heterocyclic group.

One of X¹ and X² is a single bond and the other is an oxygen atom, a sulfur atom, >NR⁷ or an optionally substituted vinylene (—CR⁸═CR⁹—) group. R⁷ is a hydrogen atom or an optionally substituted hydrocarbyl or heterocyclic group. R⁸ and R⁹ are each a hydrogen atom; a functional group containing an atom in groups 15-16 of the periodic table; a halogen atom; a hydrocarbon group optionally substituted with said functional group or a halogen atom; or an optionally substituted heterocyclic group. Note that X¹ and X² form a carbon aromatic or heterocyclic aromatic ring together with two pairs of carbon-carbon double bonds to which they are bonded.

R⁴ and R⁵ are a hydrogen atom; a functional group containing an atom in groups 15-16 of the periodic table; a halogen atom; a hydrocarbon group optionally substituted with said functional group or a halogen atom; or an optionally substituted heterocyclic group; or alternately they are bonded together to form an optionally substituted carbon or heterocyclic ring together with carbon-carbon double bonds to which they are bonded.

Z is a polymantylidene group.}

A preferable example of a compound represented by general formula (II) above is a compound represented by general formula (III) below.

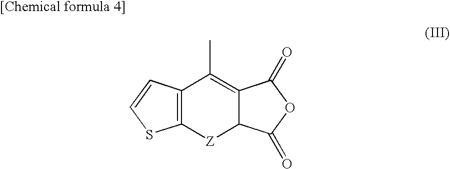

(In general formula (III), Z is a polymantylidene group.)

Among the compounds represented by general formula (III), 7-spiro-diamantane-6,7-dihydrobenzothiophene-5,6-dicarboxylic acid anhydride represented by the general formula below (IVa or IVb) is preferable.

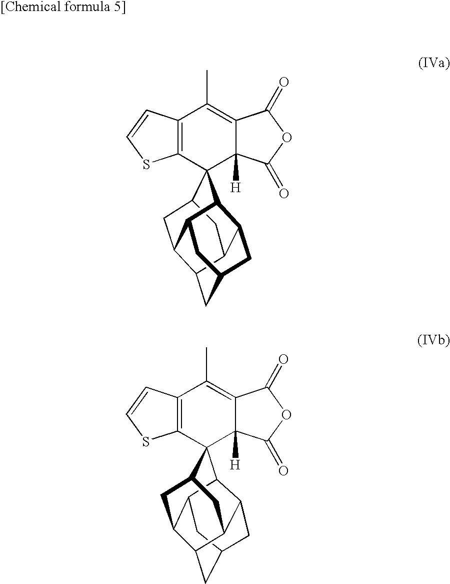

Another preferable example of a compound represented by general formula (I) is a compound represented by general formula (V).

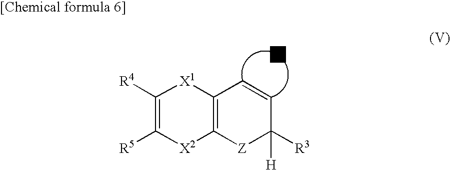

{In general formula (V), R³ is a hydrogen atom; a functional group containing an atom in groups 15-16 of the periodic table; a halogen atom; a hydrocarbon group optionally substituted with said functional group or a halogen atom; or an optionally substituted heterocyclic group.

One of X¹ and X² is a single bond and the other is an oxygen atom, a sulfur atom, >NR⁷ or an optionally substituted vinylene (—CR⁸═CR⁹—) group. R⁷ is a hydrogen atom or an optionally substituted hydrocarbyl or heterocyclic group. R⁸ and R⁹ are each a hydrogen atom; a functional group containing an atom in groups 15-16 of the periodic table; a halogen atom; a hydrocarbon group optionally substituted with said functional group or a halogen atom; or an optionally substituted heterocyclic group. Note that X¹ and X² form a carbon aromatic or heterocyclic aromatic ring together with two pairs of carbon-carbon double bonds to which they are bonded.

R⁴ and R⁵ are each a hydrogen atom; a functional group containing an atom in groups 15-16 of the periodic table; a halogen atom; a hydrocarbon group optionally substituted with said functional group or a halogen atom; or an optionally substituted heterocyclic group; or alternately they are bonded together to form an optionally substituted carbon or heterocyclic ring together with carbon-carbon double bonds to which they are bonded.

Z is a polymantylidene group.

Q is a divalent organic group that forms an optionally substituted carbon or heterocyclic ring together with the carbon-carbon double bond to which it is bonded.}

### ADVANTAGE OF THE INVENTION

The photochromic compound according to the present invention can be readily synthesized and is chemically stable, and has superior photocoloration reactivity and thermal decoloration reactivity, compared to the existing photochromic compounds.

Accordingly, the photochromic compound according to the present invention is a superior photoresponsive material and can thus be expected to have applications in automatic light-adjusting sunglasses, automatic light-adjusting window glass, photoresponsive automatic color-changing plastic materials and optical recording materials.

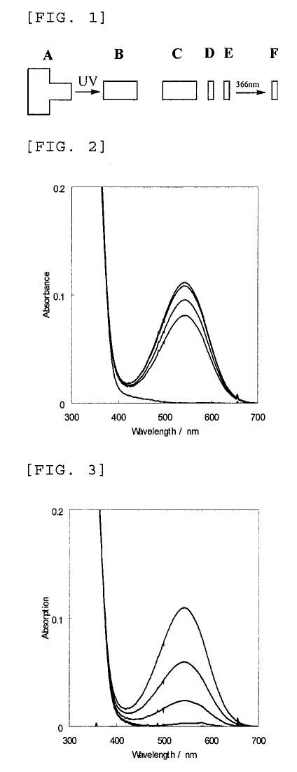

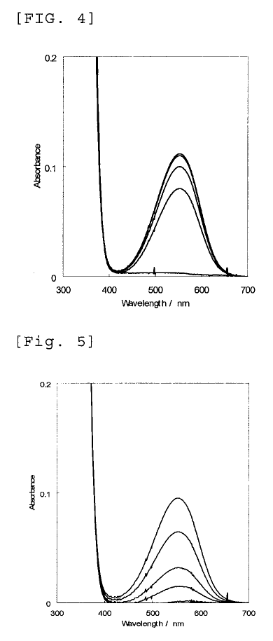

### BRIEF DESCRIPTION OF THE DRAWINGS

[FIG. 1] This is a schematic diagram of an apparatus used for irradiation of a photochromic compound with ultraviolet light.

[FIG. 2] This is a plot illustrating the change in the absorption spectrum of the photochromic compound (IV-7) according to the present invention due to an ultraviolet light-induced coloration reaction (horizontal axis: wavelength (nm), vertical axis: absorbance).

[FIG. 3] This is a plot illustrating the change in the absorption spectrum of the photochromic compound according to the present invention (IV-7) due to a thermal decoloration reaction (horizontal axis: wavelength (nm), vertical axis: absorbance).

[FIG. 4] This is a plot illustrating the change in the absorption spectrum of a known photochromic compound (R7) due to an ultraviolet light-induced coloration reaction (horizontal axis: wavelength (nm), vertical axis: absorbance).

[FIG. 5] This is a plot illustrating the change in the absorption spectrum of a known photochromic compound (R7) due to a thermal decoloration reaction (horizontal axis: wavelength (nm), vertical axis: absorbance).

### EXPLANATION OF SYMBOLS

A: 500 W ultra-high pressure mercury lamp
B: Water (Pyrex® cell: optical path length 5 cm)
C: Aqueous solution of copper sulfate (Pyrex® cell: optical path length 5 cm)
D: Colored glass filter (UV-35)
E: Colored glass filter (UV-D35)
F: Sample cell (quartz cell: optical path length 1 cm)

### BEST MODE FOR CARRYING OUT THE INVENTION

### Photochromic Compounds Represented by General Formula (I)

The photochromic compound of the present invention is represented by general formula (I).

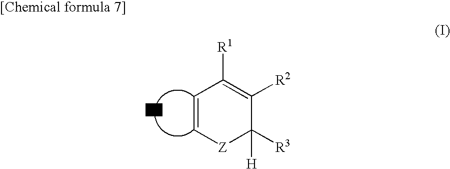

In general formula (I), R¹, R² and R³ are each a hydrogen atom; a functional group containing an atom in groups 15-16 of the periodic table; a halogen atom; a hydrocarbon group optionally substituted with said functional group or a halogen atom; or an optionally substituted heterocyclic group.

R¹ and R² as well as R² and R³ may be bonded to each other to form an optionally substituted carbon or heterocyclic ring.

Z is a polymantylidene group. In the present invention, a “polymantylidene” group refers to a divalent group with a structure obtained by removing two hydrogen atoms from one secondary carbon atom (>CH₂) of the corresponding “polymantane.”

Here, in the present invention, a “polymantane” is defined to be a compound with a higher diamondoid (also called “diamonoid”) structure represented by the general formula C₄ₙ₊₁₀H₄ₙ₊₁₆ (where n is an integer in the range 1-9). The compound with n=1 is diamantane (C₁₄H₂₀), followed by triamantane, tetramantane, . . . and the one with n=11 is dodecamantane (C₅₄H₆₀). Such compounds are known to be present in petroleum. Methods of synthesizing diamantane and tetramantane have already been established (publication of JP-A-2005-503352).

Accordingly, a diamantylidene group is a divalent group represented by general formula (VI) obtained by removing two hydrogen atoms from one secondary carbon atom (>CH₂) of diamantane (C₁₄H₂₀). Distinguishable secondary carbon atoms are present in triamantane, so there are three triamantylidene groups that can be derived therefrom.

In the present invention, a polymantylidene group may have substituents.

W is a divalent organic group that forms an optionally substituted carbon aromatic or heterocyclic aromatic ring together with the carbon-carbon double bond to which it is bonded.

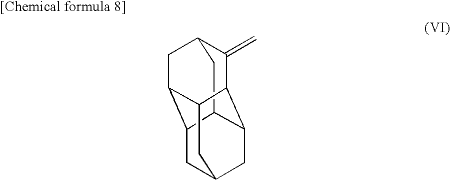

Here follows a detailed description of the photochromic compound represented by general formula (I).

In general formula (I), R¹, R² and R³ can each be a functional group containing an atom in groups 15-16 of the periodic table.

While there are no particular limitations to the atom in groups 15-16 of the periodic table, it is preferably a nitrogen atom, an oxygen atom, a phosphorus atom or a sulfur atom.

Specific examples of functional groups that contain these atoms include: amino groups, cyano groups, carbamoyl groups optionally substituted with a hydrocarbon group, nitro groups, hydroxy groups, alkoxy groups, aryloxy groups, carboxy groups, alkoxycarbonyl groups, aryloxycarbonyl groups, formyl groups, acyl groups, acyloxy groups, aroyloxy groups and others.

These functional groups may have the hydrogen atoms contained therein substituted with a hydrocarbon group.

These hydrocarbon groups may be alkyl groups or aryl groups, and the alkyl groups may be linear, branched or cyclic in form.

In general formula (I), R¹, R² and R³ can each be a halogen atom.

Specific examples of halogen atoms include a fluorine atom, a chlorine atom, a bromine atom and an iodine atom.

In general formula (I), R¹, R² and R³ can be a hydrocarbon group.

The hydrocarbon group can be an aliphatic group or an aromatic group, and the aliphatic group may be a saturated group or an unsaturated group, and may be linear, branched or cyclic in form, and if cyclic, they may be a monocyclic group or a condensed ring group. In addition, the aromatic group may be a monocyclic group or a condensed ring group.

The carbon number of the hydrocarbon group is preferably 1-20. If the hydrocarbon group is cyclic, the number of carbon atoms making up the ring is preferably 3-8.

Specific examples of the aliphatic group include: a methyl group, an ethyl group, a propyl group, an isopropyl group, a butyl group, an isobutyl group, a pentyl group, a hexyl group, a cyclopentyl group, a cyclohexyl group, a vinyl group, a propenyl group, an isopropenyl group, a cyclohexenyl group, a norbornyl group, a norbornenyl group and others.

Specific examples of the aromatic group include: a phenyl group, a naphthyl group, an anthracenyl group, a phenanthrenyl group and others.

These hydrocarbon groups may be substituted with the aforementioned functional group or a halogen atom.

In general formula (I), R¹, R² and R³ can be a heterocyclic group.

The heterocyclic group is a cyclic group containing an atom in groups 15-16 of the periodic table. There is no particular limitation as to the position of the heteroatom in the heterocyclic group. The heterocyclic group may be a monocyclic group or a condensed ring group and may be a saturated group or an unsaturated group. The number of carbon atoms making up the heterocyclic ring is preferably 3-8. In addition, there is no limitation on the number of heteroatoms contained in the heterocyclic ring.

Specific examples of the heterocyclic group include: an oxolanyl group, a dioxolanyl group, a thiolanyl group, a pyrrolidinyl group and other saturated heterocyclic groups; a furyl group, a thienyl group, a pyridyl group, an oxazolyl group, a thiazolyl group, an imidazolyl group and other unsaturated monocyclic heterocyclic groups; a benzofuryl group, a benzothienyl group, an indolyl group and other unsaturated condensed heterocyclic groups and others.

The heterocyclic group may be substituted with the aforementioned hydrocarbon group, the aforementioned functional group or a halogen atom.

In general formula (I), R¹ and R² as well as R² and R³ may be bonded to each other to form a carbon ring or a heterocyclic ring.

The carbon ring or the heterocyclic ring may be a saturated ring or an unsaturated ring, and may be monocyclic ring or a condensed ring.

The number of carbon atoms making up the carbon or heterocyclic ring is not particularly limited but is preferably 4-8. In addition, there is no limitation on the number of heteroatoms contained in the heterocyclic ring.

Specific examples of the carbon ring include: a cyclobutane ring, a cyclopentane ring, a cyclohexane ring, a norbornane ring, a cyclopentene ring, a cyclohexene ring, a cyclopentadiene ring, a cyclohexadiene ring, a norbornene ring, a benzene ring, a naphthalene ring, a phenanthrene ring and others.

Specific examples of the heterocyclic ring include: a tetrahydrofuran ring, a tetrahydrothiophene ring, a pyrrolidine ring, a dioxolane ring, a thiolane ring, a piperidine ring and other saturated heterocyclic rings; a pyrroline ring, a furan ring, a dihydrofuran ring, a dioxolene ring, a thiophene ring, a thiolene ring, a pyridine ring, an oxazoline ring, a thiazoline ring, an imidazoline ring, an oxazole ring, a thiazole ring, an imidazole ring and other unsaturated monocyclic heterocyclic ring groups; A benzofuran ring, a benzothiophene ring, an indole ring, a benzoxazole ring and other unsaturated condensed heterocyclic rings; and others.

The carbon ring or the heterocyclic ring formed by the aforementioned R¹ and R² as well as R² and R³ being bonded to each other may have substituents on the atoms constituting the rings.

The substituent may be one that replaces one hydrogen atom such as the aforementioned functional group, a halogen atoms, a hydrocarbon group, a heterocyclic group or other groups, or one that has a structure to replace two hydrogen atoms such as a carbonyl group, an imino group (═NH, ═NR) a methylene group (═CH₂) or an alkylidene group (═CR₂, ═CHR). Note that R indicates a hydrocarbon group.

Specific examples of the aforementioned R¹ and R² as well as R² and R³ that form the aforementioned carbon ring or the heterocyclic ring include: a perfluoropropano group (—(CF₂)₃—), a perfluorobutano group (—(CF₂)₄—), a cyclic acid anhydride group (—CO—O—CO—), a cyclic acid imido group (—CO—NH—CO—, —CO—NR—CO—) and others. Note that R indicates a hydrocarbon group.

Z is a polymantylidene group.

W is a divalent organic group that forms an optionally substituted carbon aromatic or heterocyclic aromatic ring together with the carbon-carbon double bond to which it is bonded.

The carbon aromatic or heterocyclic aromatic ring may be monocyclic or condensed, and the atoms constituting the rings may have substituents.

Specific examples of the carbon aromatic ring include: a benzene ring, a naphthalene ring, a phenanthrene ring and others.

Specific examples of the heterocyclic aromatic ring include: a furan ring, a thiophene ring, a pyridine ring, an oxazoline ring, an oxazole ring, a thiazole ring, an imidazole ring and other unsaturated monocyclic heterocyclic ring groups; a benzofuran ring, a benzothiophene ring, an indole ring, a benzoxazole ring and other unsaturated condensed heterocyclic rings; and others.

The substituents may be the aforementioned functional group, a halogen atom, a hydrocarbon group or a heterocyclic group.

The compound represented by general formula (I) can be obtained by the intramolecular cyclization of a compound represented by general formula (I-5) followed by 1,5-prototropy.

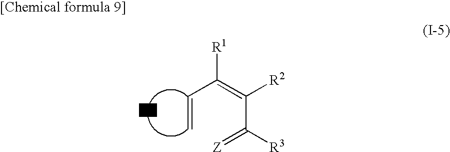

(The symbols within the formula are defined the same as in general formula (I).)

The intramolecular cyclization of the compound represented by general formula (I-5) can be performed by irradiating this compound with ultraviolet, by heating it, or by bringing it into contact with a Lewis acid catalyst.

Upon irradiation with ultraviolet light, the compound represented by general formula (I-5) is transformed into the compound indicated by general formula (I-6), and this compound instantly undergoes 1,5-prototropy with heat to change into the compound represented by generic formula (I-7). This compound is the photochromic compound indicated by general formula (I) according to the present invention. (Note that while general formula (I) is identical to general formula (I-7), a different number is applied for convenience in the explanation.)

The compound represented by general formula (I) takes the form of general formula (I-7) and is colorless at room temperature, in solution, and in a film or other matrix formed by mixing with a polymer or the like, but when irradiated with ultraviolet light, it becomes the colored compound represented by general formula (I-8).

This colored compound represented by general formula (I-8), when heated, is returned to the compound represented by general formula (I-7) and becomes colorless.

Reaction Route 1 illustrates these reactions.

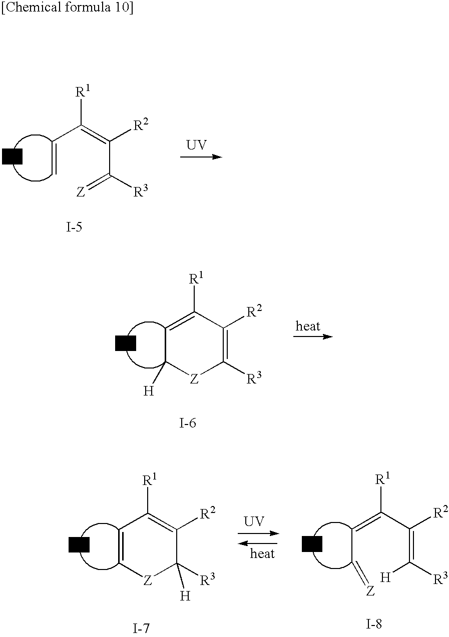

### Reaction Route 1

There are no particular limitations to the conditions for irradiation with ultraviolet light, but it is preferably performed at room temperature in a solvent.

There are no particular limitations to the solvent, as any of the following can be used: toluene, xylene or other aromatic compound solvents; hexane or other aliphatic alkane solvents; cyclohexane or other alicyclic alkane solvents; ethyl acetate or other ester solvents; N-methylpyrrolidone, N,N-dimethyl formamide or other amide solvents; tetrahydrofuran, 1,4-dioxane or other ether solvents; or others.

Among these, the aromatic compound solvents, aliphatic alkane solvents and ester solvents are preferable.

As the light source, high-pressure mercury lamps, xenon lamps and other light sources that are normally used in photochemical reactions can be used. Naturally, sunlight is also possible, and these light sources are preferably combined with appropriate wavelength filters.

The heating used to perform the intramolecular cyclization reaction of the compound represented by general formula (I-5) is preferably performed at no more than roughly 220° C., although this depends on the thermal stability of the target compound.

In addition, the Lewis acid catalyst used to perform the intramolecular cyclization reaction of the compound represented by general formula (I-5) is not particularly limited, and chlorides or bromides of aluminum, iron, tin, titanium, aluminum or the like may be used.

The amount of the Lewis acid catalyst is not particularly limited, but an amount in the range 0.001-1 mole per 1 mole of the compound represented by general formula (I-5) is preferable.

After the completion of the cyclization reaction, filtration or other operations for separation and other post-processing such as rinsing are performed, if necessary, and ultimately purification is performed by column chromatography or recrystallization to obtain the isolated target compound (I).

The compound represented by general formula (I-5) may be synthesized by any known method, but when, for example, R² and R³ are bonded to each other to form an acid anhydride group, it can be obtained by a condensation reaction of the carbonyl compound represented by general formula (I-s) with a polymantylidene succinic acid diester represented by general formula (I-2) derived from a polymantanone obtained by the oxidation of a polymantane (see Synthesis Route 1). Note that in the present invention, “polymantanone” refers to a ketone compound with a structure wherein a methylene group of a polymantylidene group is bonded to oxygen. For example, diamantanone has the structure indicated by formula (I-1a).

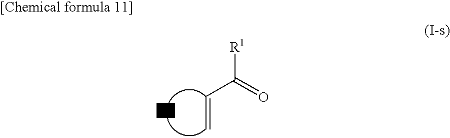

(The symbols within the formula are defined the same as in general formula (I).)

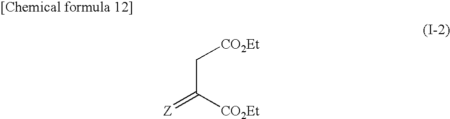

Z is a polymantylidene group.

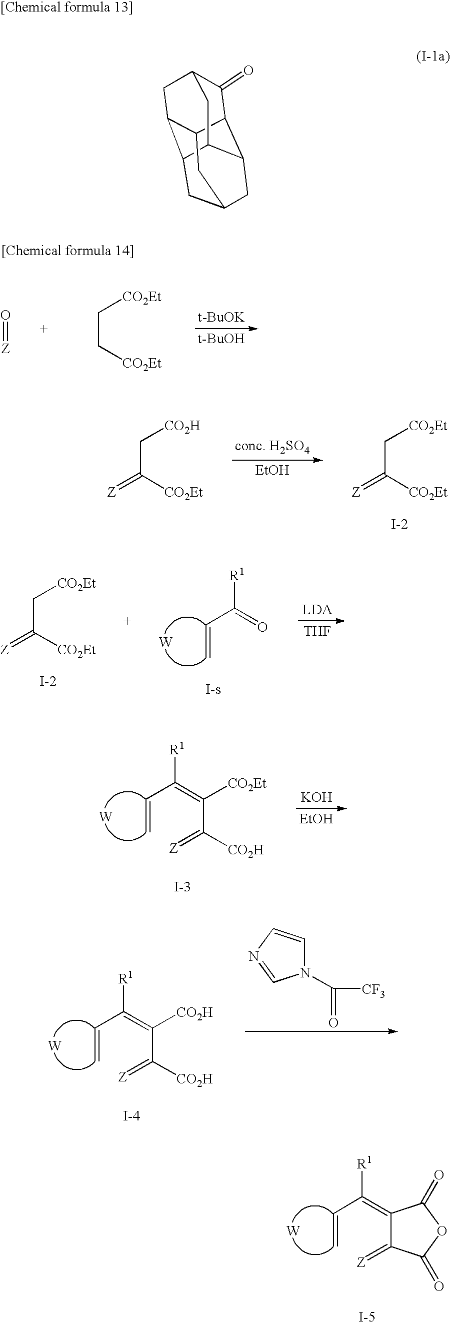

### Synthesis Route 1

### Example of Synthesis of the Compound Represented by General Formula (I-5)

In the condensation reaction of the carbonyl compound (I-s) with the polymantylidene succinic acid diester (I-2) the proportion of the carbonyl compound to the polymantylidene succinic acid ester is, as a molar ratio, 1:10 to 10:1, and preferably 1:5 to 5:1.

The reaction temperature is typically 0-100° C. and preferably 10-100° C.

The reaction is preferably performed in solution, and an aprotic solvent is preferable as the solvent. Specific examples of the solvent include: benzene, toluene and other aromatic hydrocarbon solvents, diethyl ether, tetrahydrofuran and other ether solvents.

The condensation reaction is preferably performed in the presence of a condensing agent such as sodium hydride, potassium t-butoxide, sodium ethoxide or lithium diisopropylamide. The amount of the condensing agent used is typically 0.1-10 moles per 1 mole of the carbonyl compound.

The compound represented by general formula (I-3) is converted to a free dicarboxylic acid (I-4).

This reaction can be performed by hydrolysis in the presence of a base. There are no particular limitations on the reaction conditions, but it can be performed at 0-80° C. using a 10% ethanol solution of sodium hydroxide, for example.

The dicarboxylic acid (I-4) obtained by hydrolysis is processed with a dehydrating reagent such as anhydrous acetic acid, acetyl chloride or N-trifluoroacetyl imidazole and thus converted to an acid anhydride to give the compound represented by general formula (I-5).

There is no particular limitation on the method of manufacturing a polymantylidene succinic acid diester represented by general formula (I-2), but in the case of diamantylidene succinic acid ester, the following method can be used.

To with, diamantanone is obtained by the oxidation of diamantane, and this diamantanone is condensed with diethyl succinate in the presence of a condensing agent such as sodium hydride, or potassium t-butoxide, giving diamantylidene succinic acid monoester, which is then converted to a diester using ethanol/concentrated sulfuric acid. If triamantane or tetramantane, for example, is used instead of diamantane, then the corresponding triamantylidene succinic acid ester or tetramantylidene succinic acid ester may be obtained.

### Photochromic Compounds Represented by General Formula (II)

A preferable example of the photochromic compound represented by general formula (I) is the compound represented by general formula (II) below.

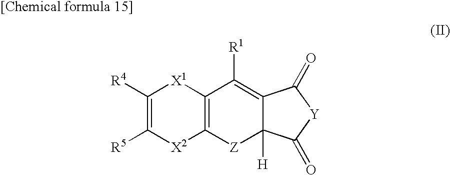

In general formula (II), R¹ is a hydrogen atom, a functional group containing an atom in groups 15-16 of the periodic table; a halogen atom; a hydrocarbon group optionally substituted with said functional group or a halogen atom; or an optionally substituted heterocyclic group.

Y is an oxygen atom, a sulfur atom or >NR⁶. R⁶ is a hydrogen atom or an optionally substituted hydrocarbyl or heterocyclic group.

One of X¹ and X² is a single bond and the other is an oxygen atom, a sulfur atom, >NR⁷ or an optionally substituted vinylene (—CR⁸═CR⁹—) group. R⁷ is a hydrogen atom or an optionally substituted hydrocarbyl or heterocyclic group. R⁸ and R⁹ are each a hydrogen atom; a functional group containing an atom in groups 15-16 of the periodic table; a halogen atom; a hydrocarbon group optionally substituted with said functional group or a halogen atom; or an optionally substituted heterocyclic group. Note that X¹ and X² form a carbon aromatic or heterocyclic aromatic ring together with two pairs of carbon-carbon double bonds to which they are bonded.

R⁴ and R⁵ are each a hydrogen atom; a functional group containing an atom in groups 15-16 of the periodic table; a halogen atom; a hydrocarbon group optionally substituted with said functional group or a halogen atom; or an optionally substituted heterocyclic group; or alternately they are bonded together to form an optionally substituted carbon or heterocyclic ring together with carbon-carbon double bonds to which they are bonded.

Z is a polymantylidene group.

In general formula (II), specific examples of aromatic rings having X¹ and X² as their constituent elements include: a furan ring, a thiophene ring, a pyrrole ring, an N-alkylpyrrole ring, a thiazole ring, a thiazoline ring and other independent 5-member heterocyclic rings; a benzofuran ring, a benzothiophene ring, an indole ring, an N-alkyl indole ring and other condensed heterocyclic rings; and a benzene ring, a naphthalene ring, a phenanthrene ring and other carbon rings.

### Method of Manufacturing Photochromic Compounds Represented by General formula (II)

The photochromic compounds represented by general formula (II) can be obtained by the intramolecular cyclization of a compound represented by general formula (II-5) followed by 1,5-prototropy (see Reaction Route 2). Note that while general formula (II) is identical to general formula (II-7), a different number is applied for convenience in the explanation.

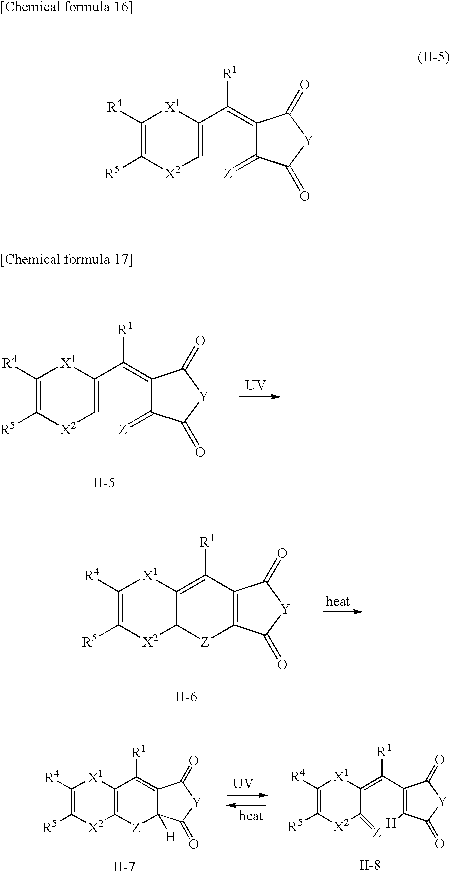

### Reaction Route 2

The compound represented by general formula (II-5), in the case in which Y=O for example, may be obtained by a condensation reaction of the carbonyl compound represented by general formula (II-s) with a polymantylidene succinic acid diester represented by general formula (I-2) (see Synthesis Route 2).

The reaction conditions and the like are the same as in the case of obtaining the compound represented by general formula (I-5) above.

The carbonyl compound represented by general formula (II-s) can be synthesized by any known method.

In addition, the compound represented by Y=NR⁶ in general formula (II-5) may be obtained by reacting the compound represented by Y=O in general formula (II-5) with the amine compound R⁶NH₂ and then performing dehydration cyclization.

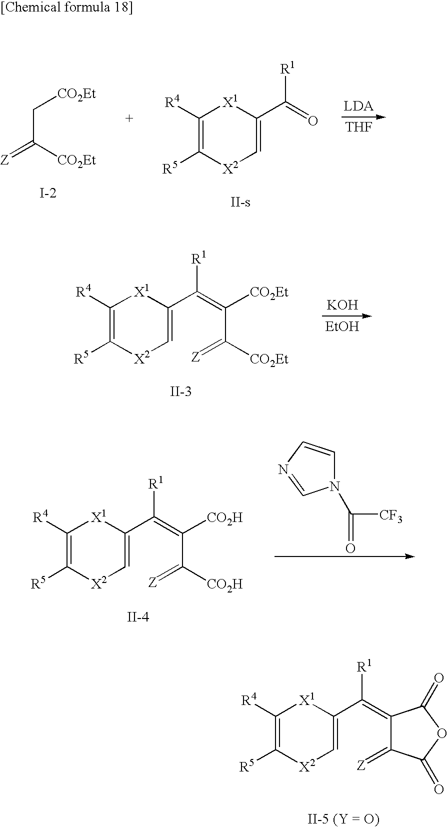

### Synthesis Route 2

### Photochromic Compounds Represented by General formula (III)

A specific preferable example of a photochromic compound represented by general formula (II) above is a compound represented by general formula (III) below.

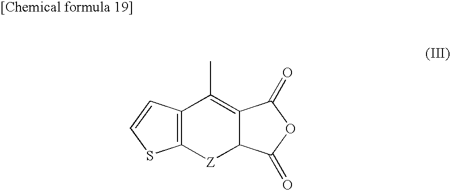

In general formula (III), Z is a polymantylidene group.

The compound represented by general formula (III) may be any one of the two diastereomers represented by the general formulae (IVa) and (IVb) below.

The compound represented by general formula (III) may be a mixture of the two diastereomers represented by the general formulae (IVa) and (IVb), and there is no particular limitation to the proportions thereof.

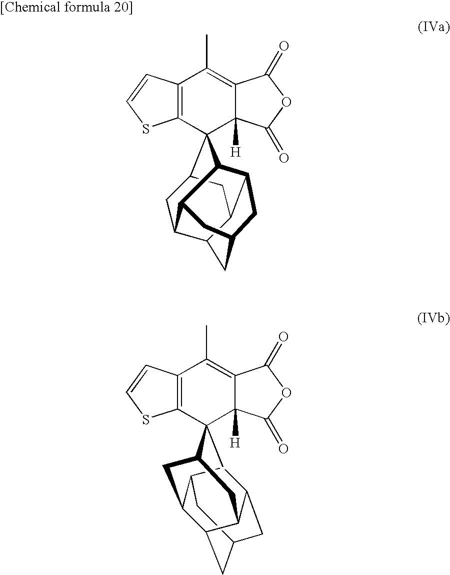

The photochromic compounds represented by general formula (III) can be obtained by the intramolecular cyclization of a compound represented by general formula (III-5) followed by 1,5-prototropy (see Reaction Route 3).

The intramolecular cyclization of the compound represented by general formula (III-5) can be performed by irradiating this compound with ultraviolet, by heating it, or by bringing it into contact with a Lewis acid catalyst.

Upon irradiation with ultraviolet light, the compound represented by general formula (III-5) is changed into the compound represented by general formula (III-6), and this compound instantly undergoes 1,5-prototropy with heat to be the compound represented by generic formula (III-7). This compound is the photochromic compound represented by general formula (III) according to the present invention. (Note that while general formula (III) is identical to general formula (III-7), a different number is applied for convenience in the explanation.)

The compound represented by general formula (III) takes the form of general formula (III-7) and is colorless at room temperature, in solution, and in a film or other matrix formed by mixing with a polymer or the like, but when irradiated with ultraviolet light, it becomes the colored compound represented by general formula (III-8).

This colored compound represented by general formula (III-8), when heated, is returned to the compound represented by general formula (III-7) and becomes colorless.

Reaction Route 3 illustrates these reactions.

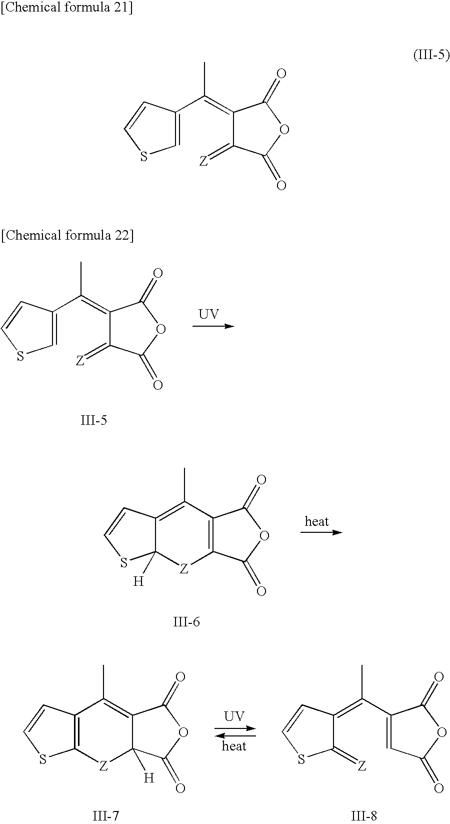

### Reaction Route 3

The photochromic compound represented by general formula (III-5) is not limited by the methods of manufacture thereof, and it can be preferably obtained from polymantanone and 3-acetylthiophene via Synthesis Route 3 below.

Here follows a description of diamantanone as an example.

To wit, diamantanone is obtained by the oxidation of diamantane.

Diamantanone is condensed with diethyl succinate to give 2-diamantylidenesuccinic acid monoester.

Next, these carboxyl groups are converted to a diethyl ester to give 2-diamantylidenesuccinic acid diester.

This 2-diamantylidenesuccinic acid diester is subjected to a condensation reaction with 3-acetylthiophene in tetrahydrofuran (THF) in the presence of lithium diisopropylamide (LDA), and via the half ester (III-3) from the product thus obtained, 2-[1-(3-thienyl)-ethylidene]-3-diamantylidenesuccinic acid (III-4) is obtained.

This 2-[1-(3-thienyl)-ethylidene]-3-diamantylidenesuccinic acid (III-4) is subjected to dehydration cyclization to be converted to the acid anhydride: 4-diamantylidene-3-[1-(3-thienyl)-ethylidene]-dihydro-2,5-furandione (III-5).

The conditions and the like for these reactions are as described above regarding the synthesis of the compound of general formula (II-5).

If triamantane or tetramantane, for example, is used instead of diamantane, then the corresponding 4-triamantylidene-3-[1-(3-thienyl)-ethylidene]-dihydro-2,5-furandione or 4-tetramantylidene-3-[1-(3-thienyl)-ethylidene]-dihydro-2,5-furandione may be obtained.

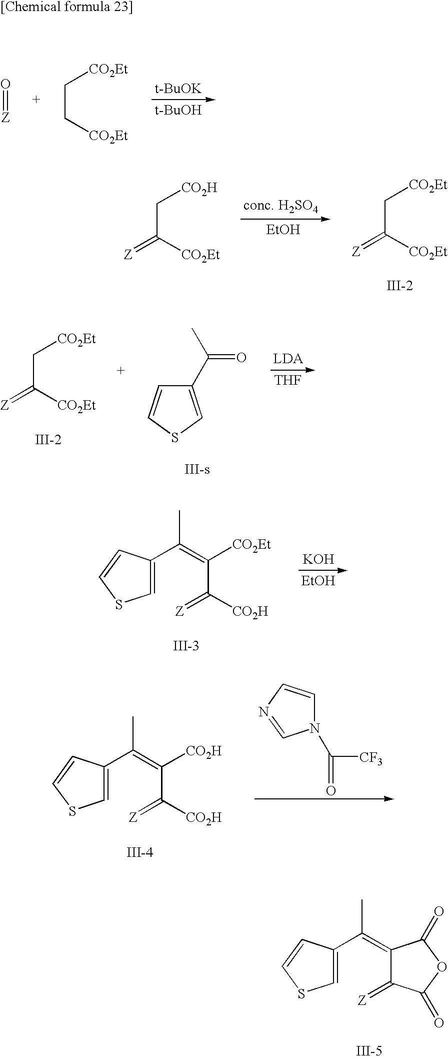

### Synthesis Route 3

### Photochromic Compounds Represented by General Formula (V)

Another preferable example of a compound represented by general formula (I) is a compound represented by general formula (V) below.

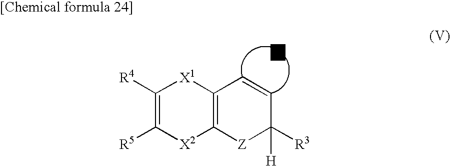

In general formula (V), R³ is a hydrogen atom; a functional group containing an atom in groups 15-16 of the periodic table; a halogen atom; a hydrocarbon group optionally substituted with said functional group or a halogen atom; or an optionally substituted heterocyclic group.

One of X¹ and X² is a single bond and the other is an oxygen atom, a sulfur atom, >NR⁷ or an optionally substituted vinylene (—CR⁸═CR⁹—) group. R⁷ is a hydrogen atom or an optionally substituted hydrocarbyl or heterocyclic group. R⁸ and R⁹ are each a hydrogen atom; a functional group containing an atom in groups 15-16 of the periodic table; a halogen atom; a hydrocarbon group optionally substituted with said functional group or a halogen atom; or an optionally substituted heterocyclic group. Note that X¹ and X² form a carbon aromatic or heterocyclic aromatic ring together with two pairs of carbon-carbon double bonds to which they are bonded.

R⁴ and R⁵ are each a hydrogen atom; a functional group containing an atom in groups 15-16 of the periodic table; a halogen atom; a hydrocarbon group optionally substituted with said functional group or a halogen atom; or an optionally substituted heterocyclic group; or alternately they are bonded together to form an optionally substituted carbon or heterocyclic ring together with carbon-carbon double bonds to which they are bonded.

Z is a polymantylidene group.

Q is a divalent organic group that forms an optionally substituted carbon or heterocyclic ring together with the carbon-carbon double bond to which it is bonded.

The photochromic compounds represented by general formula (V) can be obtained by the intramolecular cyclization of a compound represented by general formula (V-5) followed by 1,5-prototropy.

The reaction route and reaction conditions are the same as for the compound represented by general formula (I).

The compound represented by general formula (V-5) may be synthesized via Synthesis Route 5 below, for example. Note that in the formula, M is a metal atom.

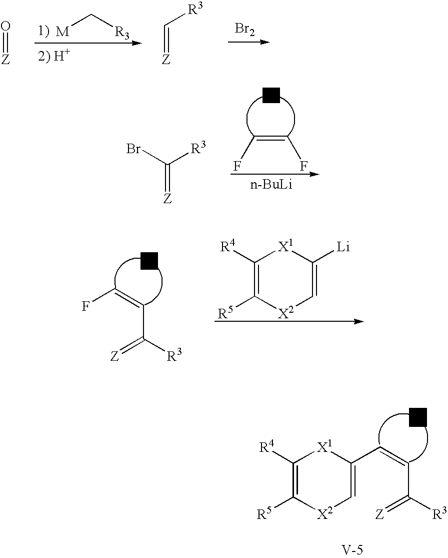

### Synthesis Route 5

By combining the photochromic compound according to the present invention with an ultraviolet stabilizer or an ultraviolet absorbent, the durability of its photochromic action can be improved. Accordingly, when the photochromic compound according to the present invention is put into practical use, it can be mixed with and used together with known ultraviolet stabilizers or ultraviolet absorbents.

A polymer that forms such a polymer solid matrix may be any one that uniformly disperses the compound according to the present invention, and optically preferable thermoplastic resins include, for example: polymethyl acrylate, polyethyl acrylate, polymethyl methacrylate, polyethyl methacrylate, polystyrene, polyacrylonitrile, polyvinyl alcohol, polyacrylamide, poly(2-hydroxyethyl methacrylate), polydimethylsiloxane, polycarbonate, poly(allyl diglycol carbonate), amorphous polyolefin or other polymers, or any polymers formed by copolymerization of these monomers as the raw materials for these polymers or copolymerization of these monomers with other monomers. There are no particular limitations on the molecular weight of these thermoplastic resins, but rather they are typically selected within the range of 500-500,000.

In addition, thermosetting resins include: polymers of radically polymerizable multifunctional monomers such as ethylene glycol diacrylate, diethylene glycol dimethacrylate, triethylene glycol dimethacrylate, tetraethylene glycol dimethacrylate, propylene glycol dimethacrylate, tripropylene glycol dimethacrylate, tetrapropylene glycol dimethacrylate, ethylene glycol bis-glycidyl methacrylate, bisphenol A dimethacrylate, 2,2-bis(4-methacryloyloxyethoxyphenyl)propane and other polyhydric acrylate and methacrylate ester compounds; diallyl phthalate, diallyl terephthalate, diallyl isophthalate, diallyl tartarate, diallyl epoxy succinate, diallyl fumarate, diallyl chlorendate, diallyl hexaphthalate, diallyl carbonate, allyl diglycol carbonate, trimethylolpropanetriallyl carbonate and other polyvalent acrylic compounds; 1,2-bis(methacryloylthiomethyl)benzene and other polyhydric thioacrylate and thiomethacrylate esters; glycidyl acrylate, glycidyl methacrylate, β-methyl glycidyl methacrylate, bisphenol A-monoglycidyl ether methacrylate, 4-glycidyloxy methacrylate, 3-(glycidyl-2-oxyethoxy)-2-hydroxypropyl methacrylate, 3-(glycidyloxy-1-isopropyloxy)-2-hydroxypropyl acrylate or other methacrylate compounds or acrylate compounds; divinyl benzene; or the like.

Additional examples include: copolymers of these monomers with radically polymerizable monomers including unsaturated carboxylic acids such as acrylic acid, methacrylic acid, maleic acid and the like; acrylate and methacrylate ester compounds such as methyl acrylate, methyl methacrylate, benzyl methacrylate, phenyl methacrylate, 2-hydroxyethyl methacrylate and the like; fumarate ester compounds such as diethyl fumarate, diphenyl fumarate and the like; thioacrylate and thiomethacrylate ester compounds such as methyl thioacrylate, benzyl thioacrylate, benzyl thiomethacrylate and the like; vinyl compounds such as styrene, chlorostyrene, methylstyrene, vinylnaphthalene, α-methylstyrene, α-methylstyrene dimer, bromostyrene and the like.

Further examples include: addition polymers of a polyhydric isocyanate compound such as xylene diisocyanate, p-phenylene diisocyanate with a polyhydric alcohol or polyhydric thiol compound such as ethanedithiol, propanetriol, hexanedithiol, pentaerythritol tetrakis(thioglycolate), ethylene glycol, trimethylolpropane, pentaerythritol, bisphenol A and the like. One or two or more of these monomers can also be mixed and used as the raw material.

Methods of dispersing the compound according to the present invention in the aforementioned polymers, when the polymer is a thermoplastic resin, include a method where the synthesis of the thermoplastic resin, namely, polymerization is performed in the presence of the compound according to the invention, and a method where the thermoplastic resin and the compound according to the invention are melt-kneaded at a temperature not lower than the melting point of the thermoplastic resin.

In the case that the polymer is a thermosetting resin, a method wherein the raw material for the thermoplastic resin is blended with the compound according to the present invention and then polymerized.

When the compound according to the present invention is to be dispersed in the aforementioned polymer, the amount of the compound according to the present invention to be added per 100 parts by weight of the polymer is typically in the range of 0.001-70 parts by weight, or preferably 0.005-30 parts by weight, or particularly preferably 0.1-15 parts by weight. In addition, in the case that the aforementioned ultraviolet stabilizer or ultraviolet absorbent is to be mixed into the polymer, the amount thereof is preferably such that the aforementioned mixture ratio between the compound according to the present invention and the ultraviolet stabilizer or ultraviolet absorbent is maintained.

### EXAMPLES

Here follows an even more specific description of the present invention made with synthesis examples and working examples. Note that the parts and percentages presented in the various examples are calculated on a mass basis unless specifically noted otherwise.

Each property is evaluated by the following method.

### Confirmation of the Structure of the Compound

This is based on ¹H-NMR using tetramethylsilane (TMS) in a deuterated chloroform (CDCl₃) solution as the standard substance.

### Example 1

### Synthesis of 7-spiro-diamantane-6,7-dihydrobenzothiophene-5,6-dicarboxylic acid anhydride (IVa-7a and 7b)

### Synthesis of 2-diamantylidene succinic acid monoester (IV-1a and 1b)

To a 50-ml pear-shaped flask, added were a spinner and diamantanone (1.1715 g, 5.79 mmol, 1.00 equivalent), and after purging with nitrogen, added were diethyl succinate (2.392 g, 2.39 equivalent) and tetrahydrofuran (THF) (13 ml) in this order followed by stirring. Note that the diamantanone used was obtained from Chevron.

To a 100-ml two-necked pear-shaped flask, added were a spinner and potassium t-butoxide (t-BuOK) (0.724 g, 6.41 mmol, 1.11 equivalent), after purging with nitrogen, added was t-butanol (t-BuOH) (12 ml) and followed by heating in an oil bath. When the t-BuOK dissolved and began to reflux, the THF solution of diamantanone and diethyl succinate prepared in the 50-ml pear-shaped flask was slowly added dropwise using a cannula. After the end of dropwise addition, the reaction mixture is allowed to reflux for approximately 5 hours and then cooled down to room temperature and water was added thereto. Here, 5M-hydrochloric acid was added to bring the pH to 1. At this time, a white precipitate was produced. This was extracted three times with diethyl ether and the organic layers thus collected were rinsed with water, and then extracted five times with 10% sodium carbonate solution. Added was 5 M-hydrochloric acid to the water layer thus collected and the pH was brought to 1. The water layer was extracted three times with diethyl ether and the organic layers thus collected were rinsed with water and then washed with a saturated saline solution, and then dried over anhydrous sodium sulfate. After separating the drying agent by filtration, 1.06 g of a half ester (mixture of IV-1a and 1b) was obtained.

### Synthesis of diethyl 2-diamantylidene succinate (IV-2a and 2b)

To a 100-ml two-necked pear-shaped flask, added were a spinner and the 2-diamantylidene succinic acid monoester (1.0552 g) containing impurities, followed by purging with nitrogen. To this, added were 15 ml of ethanol and 0.7 ml of concentrated sulfuric acid, followed by reflux for approximately 4 hours. After cooling to room temperature, was added diethyl ether. An aqueous solution of 10% sodium carbonate was added until the pH reached 8 or higher, then the reaction mixture was extracted three times with diethyl ether, then rinsed with a saturated saline solution, and dried over anhydrous sodium sulfate. After separating the drying agent by filtration, the solvent was distilled off under vacuum followed by purification by flash column chromatography (developing solvents: 2% ethyl acetate/hexane) to give 836 mg of a mixture of diethyl diamantylidene succinate (IV-2a and 2b) and diethyl succinate. From NMR, the ratio of diethyl adamantylidene succinate to diethyl succinate was estimated to be 2:1. The yield was 27%.

The ¹H-NMR data were as follows.

¹H-NMR (270 MHz) δ (ppm): 1.24 (3H, t, J/Hz=7.2), 1.28 (3H, t, J/Hz=6.3), 1.99-1.70 (17H, m), 2.66 (1H, s, one of the isomers), 2.72 (1H, s, one of the isomers), 3.36 (1H, s), 3.37 (1H, s), 3.53 (1H, s, one isomer), 3.59 (1H, s, one of the isomers), and 4.2 (4H, q, J/Hz=7.2).

### Synthesis of 2-[1-(3-thienyl)-ethylidene]-3-diamantylidene succinate (IV-4a and 4b)

To a 30-ml pear-shaped flask, added were 770 mg of the mixture of diethyl adamantylidene succinate with diethyl succinate obtained above and a spinner, and the interior of the system was purged with nitrogen. 10 ml of tetrahydrofuran (THF) was added followed by stirring, and cooled to 0° C. in an ice bath.

To another 30-ml pear-shaped flask, added were 274 mg (2.17 mmol) of 3-acetylthiophene and a spinner, and the interior of the system was purged with nitrogen. 10 ml of tetrahydrofuran (THF) was added followed by stirring and cooled to 0° C. in an ice bath.

To a 100-ml two-necked pear-shaped flask, a spinner was added followed by purging with nitrogen. Added were a small amount of 2,2′-bipyridyl, and then 0.45 ml (3.2 mmol) of diisopropylamine and 20 ml of THF, and the reactor is cooled using a freezing bath (dry ice/isopropanol; −78° C.). n-Butyl lithium (hexane solution, 1.6 mol/dm3) in an amount 2.1 ml (3.2 mmol) of was added dropwise using a syringe (at a rate of one drop/second) and then the THF solution of diethyl diamantylidene succinate was slowly added dropwise using a cannula.

After stirring for one hour, the THF solution of 3-acetylthiophene was slowly added dropwise and then the freezing bath was removed and the reaction mixture was left at room temperature to allow the temperature to raise while stirring for 16 hours. Thereafter, the reaction mixture is refluxed for eight hours, cooled and then 4M-hydrochloric acid was added to make the reaction mixture acidic and the reaction mixture was extracted four times with diethyl ether. The thus collected organic layers were rinsed with an aqueous solution of 10% sodium hydrogen carbonate and then with a saturated saline solution, and then dried over anhydrous sodium sulfate. After separating the drying agent by filtration, the solvent was vacuum-distilled off to give 915 mg of a half ester (IV-3a and 3b).

To a 100-ml two-necked pear-shaped flask, were added the half ester (IV-3a and 3b) obtained above and a spinner, and then 1.25 g (21.7 mmol) of potassium hydroxide, 15 ml of ethanol and 20 ml of water, and the mixture was refluxed for approximately 13 hours at 100-110° C. The reaction solution was cooled down to room temperature followed by addition of diethyl ether and then extracted four times with an aqueous solution of 10% sodium hydrogen carbonate. 4M-hydrochloric acid was added to the thus collected aqueous layers to adjust the pH to 1. At this time, a white precipitate was produced. This precipitate was extracted four times with diethyl ether, and the collected organic layers were rinsed with a saturated saline solution, and then dried over anhydrous sodium sulfate. After separating the drying agent by filtration, the solvent was vacuum-distilled off to give 774 mg of 3-diamantylidene-2-[1-(3-thienyl)-ethylidene]-succinate (IV-4a and 4b).

### Synthesis of 4-diamantylidene-3-[1-(3-thienyl)-ethylidene]-dihydro-2,5-furandione (IV-5a and 5b)

To a 100-ml two-necked pear-shaped flask, added were the 3-diamantylidene-2-[1-(3-thienyl)-ethylidene]-succinate (IV-4a and 4b) thus obtained and a spinner followed by purging of the system with nitrogen. 15 ml of THF was added to the reactor followed by stirring and then 0.5 ml of N-trifluoroacetyl imidazole was added at room temperature followed by stirring overnight. Water was added to the reaction solution, and then the reaction solution was extracted four times with diethyl ether and then rinsed with a saturated saline solution, and dried over anhydrous sodium sulfate. After separating the drying agent by filtration, the solvent was vacuum-distilled off and the reaction product was purified by flash column chromatography (developing solvents: 3% ethyl acetate/hexane) to give 107 mg (0.27 mmol) of 4-diamantylidene-3-[1-(3-thienyl)-ethylidene]-dihydro-2,5-furandione (IV-5a and 5b) at a yield of 13% (based on acetylthiophene).

The ¹H-NMR data were as follows.

¹H-NMR (400 MHz) δ (ppm): 1.52-1.88 (17H, m), 2.29 (1H, s), 2.32 (1H, s), 2.62 (3H, s, one of the isomers), 2.53 (3H, s, one of the isomers), 4.02 (1H, s, one of the isomers), 4.04 (1H, s, one of the isomers), 7.11 (1H, m), 7.32 (1H, m), and 7.40 (1H, m).

IR spectrum was measured with a KBr pellet. The data (v/cm) were as follows: 3103, 2916, 2887, 2867, 2847, 1804, 1755, 1753, 1616, 1608, 1226, 1212, 944, and 926.

### Synthesis of 7-spiro-diamantane-6,7-dihydrobenzothiophene-5,6-dicarboxylic acid anhydride (IV-7a and 7b)

To a 5-cm large cylindrical cell, added were 46.0 mg (0.12 mmol) of 4-diamantylidene-3-[1-(3-thienyl)-ethylidene]-dihydro-2,5-furandione and a spinner and the cell was filled with deaerated ethyl acetate. After nitrogen was bubbled through the mixture for approximately 10 minutes, the mixture was irradiated with 366 nm light for 10 hours and then the solvent was distilled off to quantitatively give (IV-7a and 7b). The ¹H-NMR measurement showed that two diastereomers (IV-7a and 7b) were produced at a ratio of approximately 2:1. It is not clear which of the diastereomers (IV-7a and 7b) was produced in a major quantity.

The ¹H-NMR data were as follows.

¹H-NMR (400 MHz) δ (ppm): Major-quantity component: 1.52-2.00 (16H, m), 2.27 (1H, s), 2.46 (1H, s), 2.66 (3H, d, J/Hz=2.2), 3.98 (1H, q, J/Hz=2.2), 7.15 (1H, d, J/Hz=5.4) and 7.23 (1H, d, J/Hz=5.4).

Minor-quantity component: 1.52-2.00 (16H, m), 2.32 (1H, s), 2.53 (1H, s), 2.65 (3H, d, J/Hz=2.2), 3.71 (1H, q, J/Hz=2.2), 7.13 (1H, d, J/Hz=5.4), and 7.21 (1H, d, J/Hz=5.6)

IR spectrum was measured with a KBr pellet. The data (v/cm) were as follows: 3106, 2915, 2900, 2886, 2849, 2360, 2342, 1813, 1811, 1757, 1646, 1253, 1204, 963, 934, and 926.

From the above results, the product was confirmed to be the aforementioned 7-spirodiamantane-6,7-dihydrobenzothiophene-5,6-dicarboxylic acid anhydride (IV-7a and 7b)

Synthesis Route 4 presents the above series of reactions.

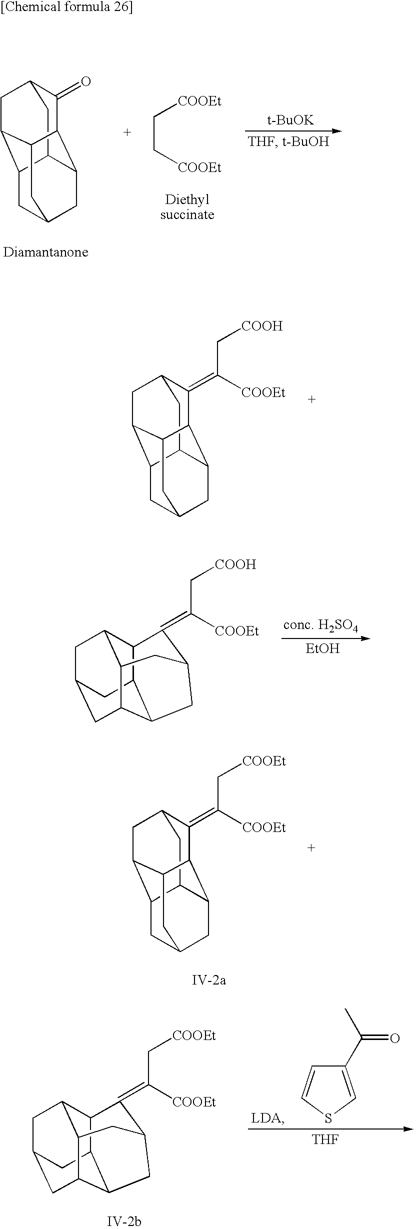

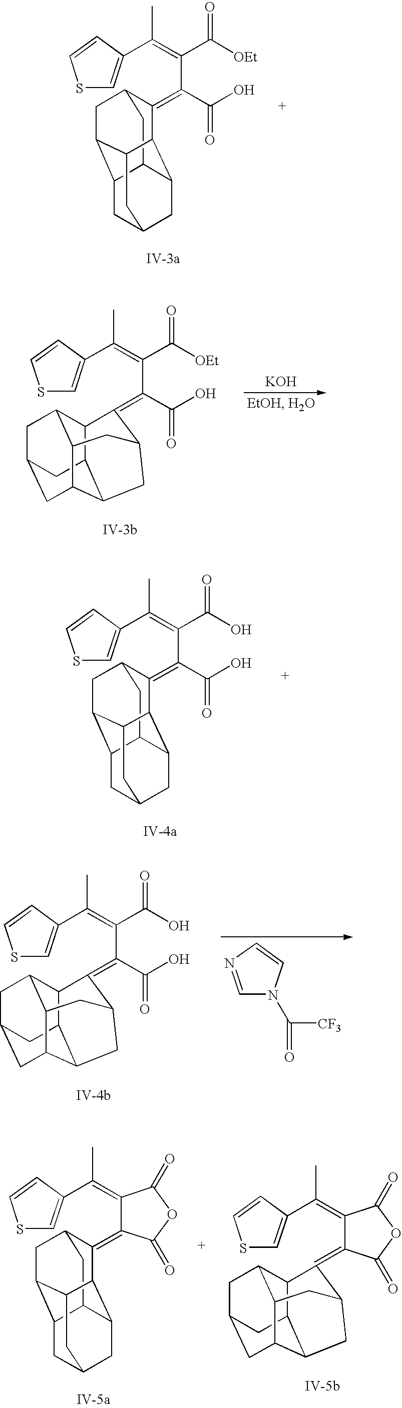

### Synthesis Route 4

The photochromic compound (IV) according to the present invention can be obtained from the compounds represented by general formulae (IV-5a) and (IV-5b) above by intramolecular cyclization followed by 1,5-prototropy (see Reaction Route 4).

### Photochromism

The compounds represented by general formulae (IV-5a) and (IV-5b) are colorless at room temperature, in solution, and in a film or other matrix formed by mixing with a polymer or the like, but when irradiated with ultraviolet light, they are cyclized to give the compounds represented by general formulae (IV-6a) and (IV-6b), which with heat instantly undergo hydrogen transfer, changing to the compounds represented by general formulae (IV-7a) and (IV-7b). The compounds represented by general formulae (IV-7a) and (IV-7b) are colorless, but upon irradiation with ultraviolet light they turn into the reddish purple compounds represented by general formulae (IV-8a) and (IV-8b).

The colored compounds represented by general formulae (IV-8a) and (IV-8b), with heat, return to the compounds represented by general formulae (IV-7a) and (IV-7b) and become colorless.

These reactions are shown in Reaction Route 4.

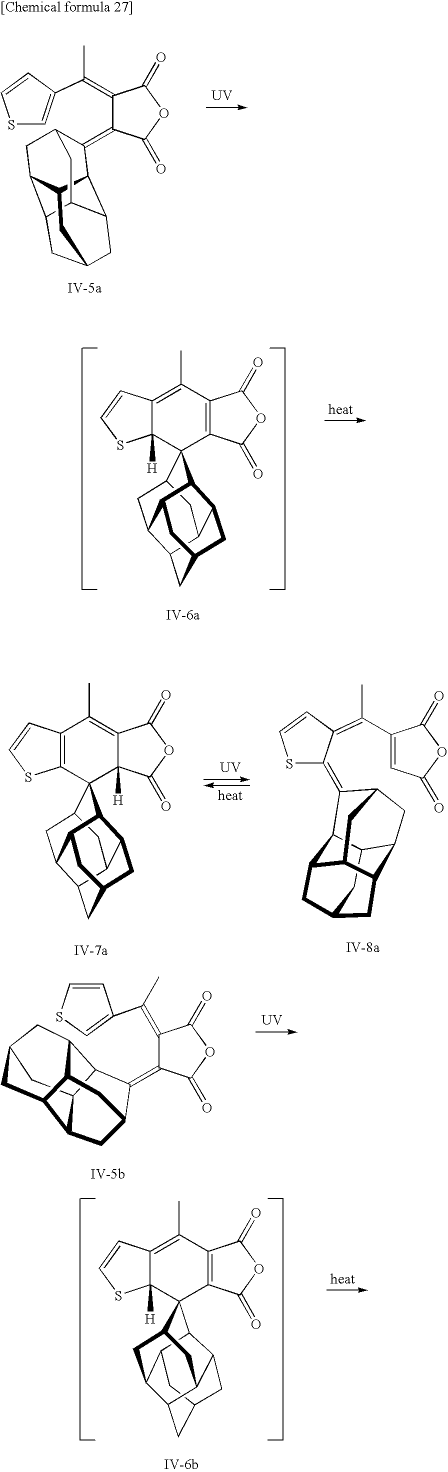

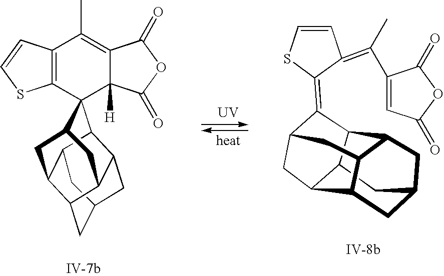

### Reaction Route 4

### Measurement of the Change in the Absorption Spectra of Compound (IV-7) Due to the Ultraviolet Light-Induced Coloration Reaction and Thermal Decoloration Reaction

A toluene solution (1.01×10⁻⁴ mol/dm³) of the approximately 2:1 mixture of compounds (IV-7a and 7b) was prepared and placed in a quartz cell with an optical path length of 1 cm and subjected to the absorption spectra measurement. For the photoreaction of compound (IV-7) to (IV-8), an ultra-high pressure mercury lamp (model USH-500D manufactured by Ushio Electric Co.) was used. The mixture was irradiated with 366-nm light brought out by the method shown in FIG. 1 for 0, 1, 2, 4 and 8 minutes. An infrared/visible light spectroscope (the “Multispec 1500 Photodiode Array Spectroscope” manufactured by Shimadzu Scientific Instruments, Inc.) was used to measure the absorption spectra for each radiation period at room temperature. During the thermal reaction from (IV-8) to (IV-7), the cell was set and left in the spectroscope at room temperature and the absorption spectra after 0, 1, 3, 5 and 10 minutes were measured.

The change at this time is illustrated in FIG. 2 (change from compound (IV-7) to (IV-8)) and FIG. 3 (change from (IV-8) to (IV-7)). Note that the maximum absorption wavelength was 553.0 nm.

In FIG. 2, the curves illustrate the absorption spectra after 0, 1, 2, 4 and 8 minutes of irradiation, in the order from bottom to top.

In addition, in FIG. 3, the curves illustrate the absorption spectra after 0, 1, 3, 5 and 10 minutes left, in the order from top to bottom.

### Comparative Example 1

A compound represented by the general formula (R-5) below (hereinafter referred to as “compound R5”) was obtained by the method recited in JP-A-S60-155179 (page (6) example 2) in a yield of 12% based on 3-acetylthiophene.

The compound R5 in an amount of 50.1 mg was dissolved in approximately 50 ml of deaerated ethyl acetate in a cell made of Pyrex® glass. The sample was irradiated with ultraviolet light with a wavelength of 366 nm for 18 hours. Thereafter, the solvent was evaporated and the sample was isolated and purified by silica gel flash column chromatography to give 33.0 mg of the compound R7 as a soft crystal.

The ¹H-NMR data were as follows.

¹H-NMR (400 MHz) δ (ppm): 60-2.00 (10H, m), 2.28 (1H, s), 2.54 (1H, s), 2.64 (3H, d, J/Hz=2.8), 3.05 (1H, d, J/Hz=10.5), 3.41 (1H, d, J/Hz=14.5), 3.99 (1H, s), 7.14 (1H, d, J/Hz=5.3), and 7.23 (1H, d, J/Hz=4.6)

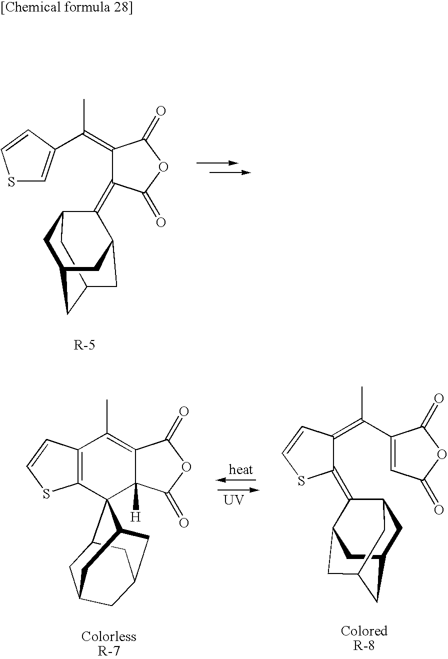

The compound R7 was dissolved in toluene to prepare a solution with a concentration of 1×10⁻⁴ mol/dm³.

In the same manner as for the solution of the compound (IV-7) of Example 1, the change in the absorption spectra of this solution due to the ultraviolet light-induced coloration reaction and thermal decoloration reaction was measured.

From FIGS. 2-5, it is observed that, with the compound (IV-7) in toluene, coloration up to the maximum intensity of 77% occurred within 1 minute from the start of ultraviolet radiation, while with the compound R7, only 70% was reached 1 minute from the start of ultraviolet radiation.

On the other hand, with respect to the decoloration rate after ultraviolet radiation was halted, the absorbance dropped to 49% after 1 minute and 12% after 3 minutes and is completely decolored in approximately 5 minutes for compound (IV-7), while the absorbance dropped only to 67% after 1 minute and 33% after 3 minutes, requiring approximately 10 minutes to be completely decolored for the compound R7.

These results are thought to be because the presence of bulky polymantylidene makes the excitation state of the compound (IV-7) under ultraviolet radiation more stable than that of the adamantylidene group of the compound R7, so the shift to the compound (IV-8) is faster and coloration becomes stronger, but without ultraviolet radiation, compound (IV-8) is more unstable and returns to compound (IV-7) more quickly.

From this, it can be understood that the rate of coloration under ultraviolet radiation is faster for the photochromic compound according to the present invention than for the known photochromic compound (R7), thus reaching a light-heat steady state more quickly, and the decoloration rate is also faster.

Thus, the photochromic compound according to the present invention is suitable for application to photochromic materials.

### INDUSTRIAL UTILITY

As the compound according to the present invention exhibits excellent photochromic properties, it is useable for photochromic lens materials, optical filter materials, display materials and materials for use in photometers and for decorative uses.

Examples of methods of using the photochromic compound according to the present invention in a photochromic lens include: a method by which the photochromic compound according to the present invention is dispersed uniformly within a polymer film which is sandwiched within the lens, or a method wherein the photochromic compound according to the present invention is dissolved in silicone oil or the like, and this is used to impregnate the lens surface for 10-60 minutes at 150-200° C., and furthermore the surface is covered with a curable substance to form a photochromic lens, among other methods.

Moreover, a polymer containing the aforementioned photochromic compound may be used to coat the lens surface and this surface may be covered with a curable substance to form a photochromic lens. In addition, it is also possible to disperse the compound according to the present invention in advance within a polymer that can form an organic lens, which is then polymerized and hardened to form a photochromic lens.

1. A photochromic compound represented by general formula (I): 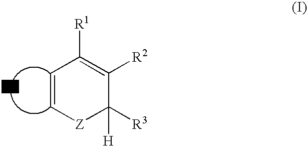
wherein:
R¹, R² and R³ are each independently a hydrogen atom; a functional group containing an atom in groups 15-16 of the periodic table; a halogen atom; a hydrocarbon group optionally substituted with said functional group or a halogen atom; or an optionally substituted heterocyclic group; or.
R¹ and R² as well as R² and R³ may be bonded to each other to form an optionally substituted carbon or heterocyclic ring;
Z is a polymantylidene group; and
W is a divalent organic group that forms an optionally substituted carbon aromatic or heterocyclic aromatic ring together with the carbon-carbon double bond to which it is bonded.

2. The photochromic compound according to claim 1 represented by general formula (II): 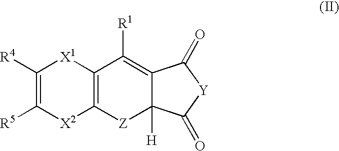
wherein:
R¹ is a hydrogen atom; a functional group containing an atom in groups 15-16 of the periodic table; a halogen atom; a hydrocarbon group optionally substituted with said functional group or a halogen atom; or an optionally substituted heterocyclic group;
Y is an oxygen atom, a sulfur atom or NR⁶. R⁶, wherein is a hydrogen atom or an optionally substituted hydrocarbyl or heterocyclic group,
One of X¹ and X² is a single bond and the other is an oxygen atom, a sulfur atom, NR⁷ or an optionally substituted vinylene (—CR⁸═CR⁹—) group, wherein R⁷ is a hydrogen atom or an optionally substituted hydrocarbyl or heterocyclic group; R⁸ and R⁹ are each independently a hydrogen atom; a functional group containing an atom in groups 15-16 of the periodic table; a halogen atom; a hydrocarbon group optionally substituted with said functional group or a halogen atom; or an optionally substituted heterocyclic group;
R⁴ and R⁵ are each independently a hydrogen atom; a functional group containing an atom in groups 15-16 of the periodic table; a halogen atom; a hydrocarbon group optionally substituted with said functional group or a halogen atom; or an optionally substituted heterocyclic group; or alternately they are bonded together to form an optionally substituted carbon or heterocyclic ring together with carbon-carbon double bonds to which they are bonded; and
Z is a polymantylidene group.

3. The photochromic compound according to claim 2 represented by general formula (III): 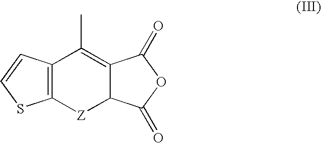
wherein:
Z is a polymantylidene group.

4. The photochromic compound according to claim 3 represented by general formula (IVa) or general formula (IVb):

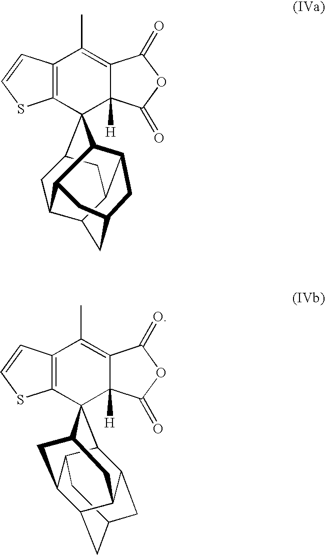

5. The photochromic compound according to claim 1 represented by general formula (V): 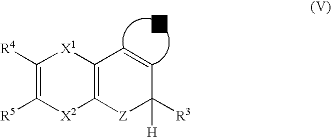
wherein:
R³ is a hydrogen atom; a functional group containing an atom in groups 15-16 of the periodic table; a halogen atom; a hydrocarbon group optionally substituted with said functional group or a halogen atom; or an optionally substituted heterocyclic group;
One of X¹ and X² is a single bond and the other is an oxygen atom, a sulfur atom, >NR⁷ or an optionally substituted vinylene (—CR⁸═CR⁹—) group. R⁷ is a hydrogen atom or an optionally substituted hydrocarbyl or heterocyclic group. R⁸ and R⁹ are each a hydrogen atom; a functional group containing an atom in groups 15-16 of the periodic table; a halogen atom; a hydrocarbon group optionally substituted with said functional group or a halogen atom; or an optionally substituted heterocyclic group. Note that X¹ and X² form a carbon aromatic or heterocyclic aromatic ring together with two pairs of carbon-carbon double bonds to which they are bonded;
R⁴ and R⁵ are each a hydrogen atom; a functional group containing an atom in groups 15-16 of the periodic table; a halogen atom; a hydrocarbon group optionally substituted with said functional group or a halogen atom; or an optionally substituted heterocyclic group; or alternately they are bonded together to form an optionally substituted carbon or heterocyclic ring together with carbon-carbon double bonds to which they are bonded;
Z is a polymantylidene group; and
Q is a divalent organic group that forms an optionally substituted carbon or heterocyclic ring together with the carbon-carbon double bond to which it is bonded.
